(19) United States
(12) Patent Application Publication
(10) Pub. No.: US 2024/0285204 A1
(43) Pub. Date: Aug. 29, 2024

(54) Method and system for automatically assigning a behavioral category to a student's study

(71) Applicant: SIT Programming School, Schaffhausen (CH)

(72) Inventors: Sergey Shedov, Madrid (ES); Ivan Dobrosovestnov, Belgrade (RS); Daniil Imani, Belgrade (RS); Semen Obukhov, Belgrade (RS); Serg Bell, Singapore (SG); Stanislav Protasov, Singapore (SG)

(21) Appl. No.: 18/175,550

(22) Filed: Feb. 28, 2023

(51) Int. Cl. A61B 5/16; G09B 19/00

(52) U.S. Cl. CPC A61B 5/165; G09B 19/00

(57) ABSTRACT

A method and system for automatically assigning a behavioral category to a subject's study progress corresponding to a particular test with an option to submit multiple versions of a solution. The method is based on abstracting subject solution versions into abstractions that are mapped to previously identified solution abstraction clusters based on some solution abstraction clustering criteria; calculating subject's path through these solution abstraction clusters and identifying a previously identified behavioral cluster to which subject's path is mapped using certain path clustering criteria. As the progress of a subject's solutions through a period of time reflects the evolution of the subject's understanding of the subject, attribution of the subject's path to a behavioral cluster allows for automatic classification of such understanding including identification of abuse or misuse of the educational process.

### FIELD OF THE INVENTION

The present disclosure generally relates to the field of education. In particular, method and system for classification of subject's progress in solving a complex problem through a period of time with an option to submit multiple versions of a solution to a given problem.

### BACKGROUND

In recent years, a large portion of the educational process moved from physical brick and mortar classrooms to online resources like virtual classrooms. With such an advancement in the field of education, an important requirement that has consequently developed is that of testing the participant subject, for example, a student or a job applicant. As a result, testing of subjects has been automated in different areas of education. There are different kinds of tests that are used nowadays. Some are short quizzes that the teachers resort to for checking subject understanding of the subjects during live lectures. The simplest form of automated testing is multiple-choice tests. Some other tests are designed to be more comprehensive and may require for the subject to provide more than a simple answer, for example, input each step of solving a mathematical, chemical, physical problem, or to write computer source code. In some tests, it is expected that subjects submit multiple versions of their solutions overtime before solving the problem.

A similar problem arises with the need to test job applicant's skills in a complex subject such as a software development process during the interviewing process.

Conventional testing systems collect and display results of all subjects who took a particular test. However, these conventional systems fail to provide the analysis of the solutions provided by each subject. More specifically, the fact that a subject ultimately solved a problem with a 100% score does not necessarily mean that the subject understands the subject. There are numerous scenarios in which a subject can provide a complete solution for a problem without understanding the subject of the problem, for example, by copying from other subjects, receiving assistance from instructors, or using other resources on the Internet.

Currently, only an instructor in personal communication with the subject can provide a true assessment of the subject's knowledge of a subject by reviewing the subject's notes and all the steps the subject took while solving a complex problem. This manual approach may be subjective and requires a significant amount of time making it in many cases prohibitively expensive.

A system and method are needed to allow for automated evaluation of a subject's progress in understanding a complex topic through the analysis of the evolution of the subject's solutions to a given problem submitted over time and its comparison to behavior of other subjects that have been previously evaluated.

### SUMMARY OF THE INVENTION

The present invention discloses a method for automatically assigning a behavioral category to a subject's study progress corresponding to a particular test. Although a student is the typical subject of the method, the method may also be used for other subjects who are not students.

The present invention relies on the method of two-level clustering of digital representations of subject solutions to a given problem.

First level is mapping of actual abstractions, for example, graphs, of versions of a subject's solution over time into previously identified solution abstraction clusters.

An example of an abstraction of a solution that is a software program is a control flow graph (CFG) which is the graphical representation of control flow or computation during the execution of programs or applications. Control flow graphs represent the flow inside of a program unit.

The second level is mapping the path of abstractions, for example, graphs, of versions of a subject's solution to a previously identified behavioral cluster.

In an embodiment, such behavioral clusters are assigned labels reflecting certain behavior types indicating, for example, that subjects whose path through solution clusters fell into a given behavioral cluster successfully understood the subject, or obtained an external help in developing a solution, or lack understanding of the subject or at least one of its components.

### DESCRIPTION OF THE DRAWINGS

FIG. 1 illustrates a system for classification of study progress of a subject in solving a complex problem, in accordance with an embodiment of the present disclosure.

FIG. 2A illustrates a step of mapping of an abstraction, for example, a graph, of a version of a subject's solution to a solution abstraction cluster.

FIG. 2B illustrates a step of mapping multiple abstractions, for example, graphs, of versions of a subject's solution to solution abstraction clusters over time.

FIG. 3 illustrates a step of mapping a subject's path to a behavioral cluster.

FIG. 4 illustrates a step of generation of a subject's path through solution abstraction clusters over time.

### DETAILED DESCRIPTION

The present invention discloses a method and a system for automatically assigning a behavioral category to a subject's progress corresponding to a particular test. The examples of a subject are a subject or a job applicant submitting multiple versions of a solution to the given test.

In accordance with the present disclosure, the method includes collection of multiple solution versions from a subject throughout a period of time.

In an embodiment, the period of time is determined by the teacher, selected by the subject, or pre-set by the designers of the test or the educational institution. For example, the period of time is several minutes, several hours, several days, a semester, or several semesters. The purpose of the test is to provide a comprehensive understanding of the subject's progress in a given subject.

Each solution version that is collected by the solution version collector is then abstracted. For example, a graph corresponding to that solution version is calculated by the solution version abstractor to generate a solution abstraction.

Each abstraction, for example, a graph, of the version of the subject's solution is mapped to a previously identified cluster according to a solution abstraction clustering criterion, for example, graph clustering criteria.

In an embodiment, if a solution abstraction, for example, a graph, is not mapped to any pre-existing solution abstraction cluster, a new solution abstraction cluster is created that includes that solution abstraction, for example, the graph, wherein the newly formed solution cluster is used in the future for solution clustering of the solution abstractions, for example, graphs.

In an embodiment, if a solution abstraction, for example, a graph, is not mapped to any pre-existing solution abstraction cluster, that solution abstraction, for example, the graph, is excluded from further analysis.

Since multiple solution versions are created and submitted by the subject, it is possible to track the progress of the subject in understanding the subject over a period of time. More specifically, the test question can be complex and may be designed in a manner that as a new lesson is taught to the subject, the solutions submitted by the subject may also require an update in accordance with the latest lessons. As a result, the analysis of all of the solution versions submitted by the subject can be used to build a path for the individual subject, wherein the subject's path is an ordered sequence of at least one solution abstraction cluster to which the abstractions, for example, graphs, of versions of subject's solution are mapped. Such a path reflects a subject's progress during the period of time.

In accordance with the present disclosure, after the path for the subject has been built, the path of the subject is mapped to a behavioral cluster based on a path clustering criterion. Thus, the system and method, as disclosed in the present disclosure, facilitate obtaining a comprehensive understanding of the progress of the subject throughout a period of time, which in one example may be one semester.

The method steps comprise collecting at least one solution version of the subject to a given test; automatically abstracting, for example, generating a graph for each version of the solution, the plurality of solution versions developed by the subject in response to the given problem; for each abstraction, for example, a graph, of the solution versions, identifying a solutions cluster into which the abstraction, for example, a graph, of that solution version belongs using at least one solution abstraction clustering criteria; building a path for the subject where path is an ordered sequence of at least one solution abstraction cluster to which at least one of the abstractions, for example, graphs, of versions of subject's solution belong; and identifying, a previously identified behavioral cluster to which the subject's path belongs using at least one path clustering criteria.

In an alternative embodiment, the behavioral clusters include ‘subject stayed in cluster for too long’, ‘subject moves from one cluster to another often’, and ‘most solutions of the subject lay in an incorrect cluster, but the correct solution is another less numerous cluster’, ‘a subject copied the solutions and does not understand the subject’, ‘a subject does not understand the subject and writes wrong code’, ‘a subject applied a different approach or approaches to solve the problem’, ‘a subject used one approach to solve the problem’, or ‘unknown’.

In an embodiment, the method further comprises using a machine learning artificial intelligence system trained on previously classified solution clusters to map a solution abstraction, for example, a graph, to a solution abstraction cluster.

In an embodiment, the step of mapping a solution abstraction, for example, a graph, to a solution abstraction cluster includes the step of using an expert artificial system.

In an alternative embodiment, the step of mapping a subject's path to a behavioral cluster comprises using a machine learning artificial intelligence system trained on previously classified behavioral clusters.

In an embodiment, the method mapping a subject's path into a behavioral cluster includes the step of using an expert artificial system.

In an embodiment, the method mapping of a solution version abstraction, for example, a graph, to a solution abstraction cluster includes at least one of a Density-Based Spatial Clustering of Applications with Noise (DBSCAN) algorithm and a k-nearest neighbors (KNN) algorithm.

In an embodiment, the method further comprises creating a new solution abstraction cluster if a given abstraction, for example, a graph, of a solution version is not mapped to any of the existing solution abstraction clusters.

In an embodiment, the method further comprises creating a new behavioral cluster if the subject's path is not mapped to any of the existing behavioral clusters.

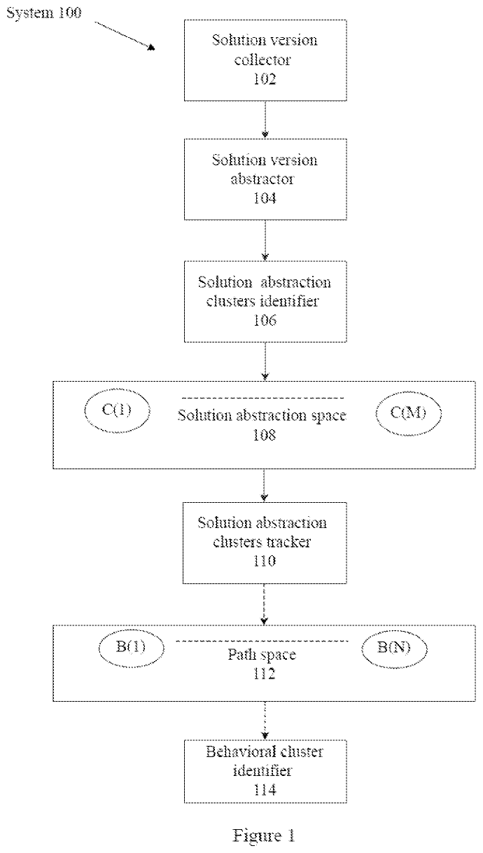

FIG. 1 illustrates a system for automatic classification of study progress of a subject in solving a complex problem 100. In accordance with an embodiment, the system 100 comprises a solution version collector 102 configured to collect different versions of a subject's solution to a given problem over time. In an embodiment, each solution version is assigned an identifier associated with the version and the subject.

In accordance with an embodiment of the present disclosure, the system 100 further comprises a solution version abstractor 104. The solution version abstractor 104 is configured for automatically abstracting each collected solution version into a solution abstraction, for example, into a graph.

The system 100 further comprises a solution clusters identifier 106 that is configured to map a subject's solution version abstraction to an existing solution abstraction cluster from a plurality of solution abstraction clusters grouped over the solution abstraction space 108 based on at least one clustering criteria. In an embodiment, the system 100 further comprises a storage that preserves metadata of solution versions, abstractions, for example, graphs, of solution versions, and their mapping to solution abstraction clusters.

In an embodiment, the system 100 further comprises a solution abstraction cluster tracker 10. The solution abstraction clusters tracker 110 is configured for creating a path of solution version abstractions, for example, graphs, of a given subject through solution abstraction clusters over time. Such mapping is required to build a path for the subject, wherein the subject's path is an ordered sequence of at least one solution abstraction cluster to which the subject's version solution abstraction, for example, a graph, is mapped. Solution abstraction clusters tracker creates a path in the path space 112.

The path of the subject generated by the solution abstraction tracker 110 reflects the subject's progress and behavior throughout the period of time.

The system 100 further comprises a behavioral clusters identifier 114 behavioral cluster configured map subject's path to an existing behavioral cluster using at least one path clustering criteria.

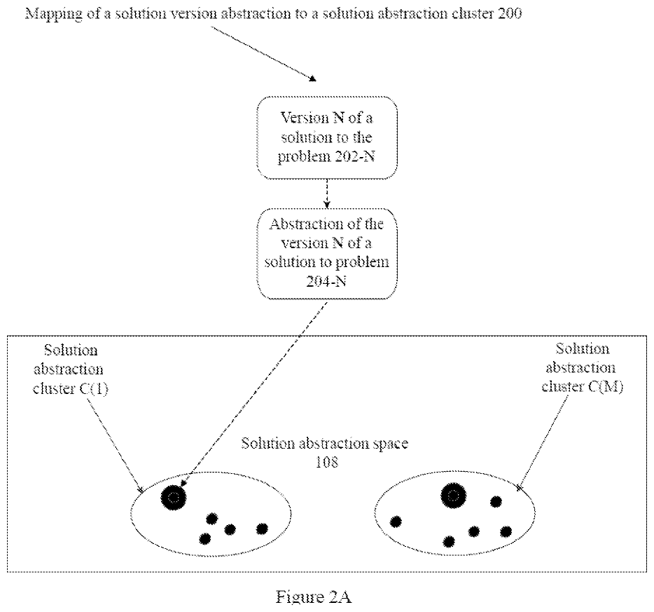

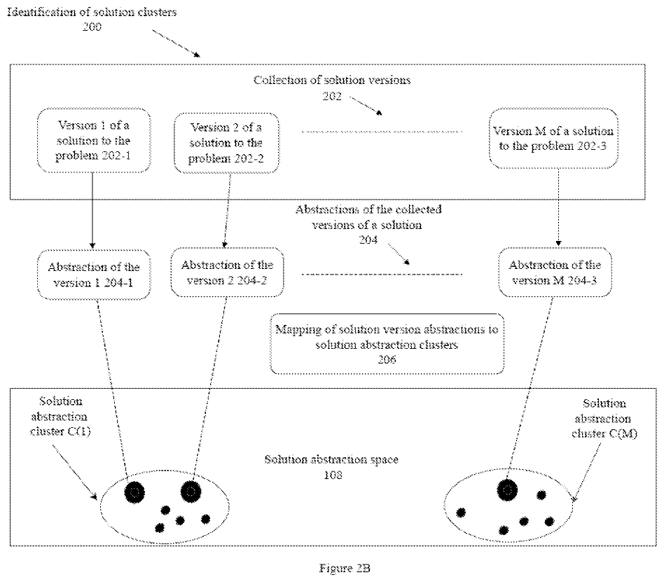

FIG. 2A illustrates step 200 of solution abstraction cluster identification in a method for classification of study progress of a subject in solving a complex problem. In accordance with an embodiment of the present disclosure, the method for classification of study progress for a subject includes collection of solution versions from the subject. The block diagram illustrated in FIG. 2A depicts the collection, abstraction, for example, creation of a graph, and mapping to a solution abstraction cluster of one solution version from the subject.

At block 202-N, the step 200 for identification of solution clusters includes receiving a solution version N for the subject as a solution to a problem. In an embodiment, each solution version is assigned an identifier associated with the version and the subject.

At block 204-N, the step 200 for identification of solution clusters includes calculating an abstraction, for example, a graph, of the solution version N of the subject. In an embodiment, the solution version abstractor 104 is configured for automatically abstracting each collected solution version into a solution abstraction, for example, into a graph.

The solution abstraction, for example, a graph, is then mapped to the solution abstraction cluster within the abstraction space of the versions 108 using at least one clustering criteria.

In an embodiment, the mapping of the solution version abstraction, for example, a graph, into at least one solution abstraction cluster is based on at least one solution abstraction clustering criteria performed by the solution abstraction cluster identifier 106.

FIG. 2B illustrates a step of solution cluster identification 200 for multiple solution versions in a method for classification of study progress of a subject in solving a complex problem, in accordance with an embodiment of the present disclosure. The block diagram illustrated in FIG. 2B depicts the collection of multiple solution versions from the subject, calculation of abstractions, for example, graphs, of these versions, and mapping of solution version abstractions, for example, graphs, to solution version clusters.

At block 202, the step 200 for identification of solution clusters includes receiving multiple solution versions 202-1, 202-2, . . . , 202-3 from the subject as multiple solution versions 1, 2, . . . , M to the given test. The multiple solution versions 202-1, 202-2, . . . , 202-3 together from a collection of solution versions 202 by the solution version collector 102. In an embodiment, each solution version is assigned an identifier associated with the version and the subject.

At blocks 204-1, 204-2, . . . , 204-3, the step 200 for identification of solution clusters includes calculating abstractions, for example, graphs, of the solution versions 1, 2, . . . , M of the subject by the solution version abstractor 104.

The method further comprises a step of mapping solution versions abstractions, for example, graphs, from the abstraction space 108 to solution abstraction clusters using at least one solution abstraction clustering criteria, for example, a graph clustering criterion, by the solution clusters identifier 106.

In an embodiment, the solution clustering criteria is based on at least one solution abstraction metric, for example, a graph metric such as.

In an embodiment, if the solution cluster identifier 106 fails to map an existing solution abstraction cluster to a solution version abstraction, for example, a graph, that solution version abstraction becomes a new solution abstraction cluster.

In an embodiment, if the solution cluster identifier 106 fails to map an existing solution abstraction cluster to a solution version abstraction, for example, a graph, that solution version abstraction is excluded from further analysis.

In an embodiment, the mapping of a solution version abstraction, for example, a graph, into solution abstraction clusters is performed using either an expert artificial intelligence system or a machine learning artificial intelligence system trained on previously analyzed mapping of solution version abstractions, for example, graphs, to solution abstraction clusters by the solution clusters identifier 106.

In an embodiment, the step of mapping of a solution version abstraction, for example, a graph, to a solution abstraction cluster includes the usage of at least one of a Density-Based Spatial Clustering of Applications with Noise (DBSCAN) algorithm or a k-nearest neighbors (KNN) algorithm.

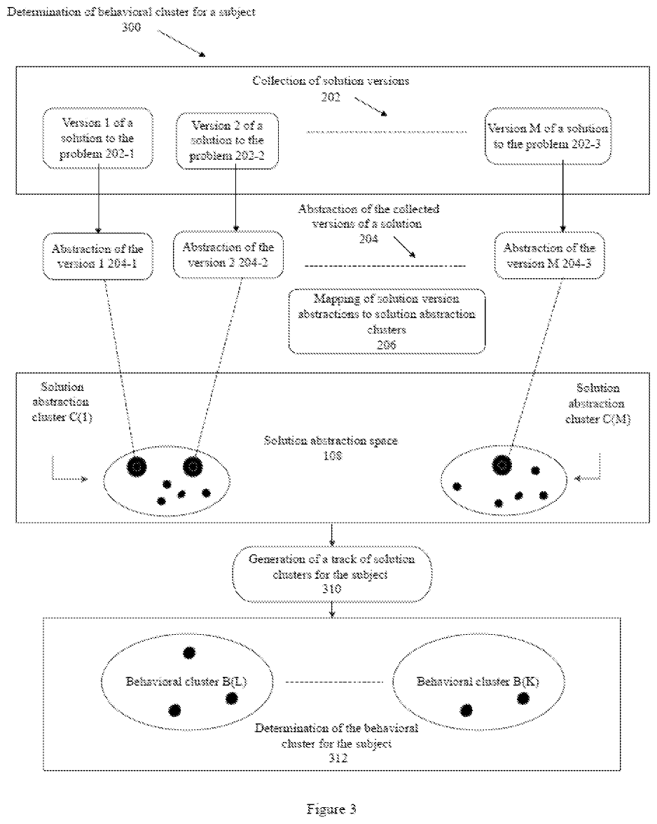

FIG. 3 illustrates step 300 of behavioral cluster identification in a method for classification of study progress of a subject in solving a complex problem, in accordance with an embodiment of the present disclosure. The step 300 for determination of a behavioral cluster includes the step 200 explained with reference to FIGS. 2A and 2B. As such FIG. 3 includes elements of FIG. 2B.

After the solution version abstractions, for example, graphs, are mapped to relevant solution abstraction clusters C(1), . . . , C(M) by the solution abstraction cluster identifier 106 over the solution abstraction space 108, the step 300 includes, at block 310, building a path for the subject, wherein the subject's path is an ordered sequence of at least one solution abstraction cluster to which the subject's solutions version abstractions, for example, graphs, belong by the solution abstraction cluster tracker 110.

In an embodiment, the system 100 includes a behavioral cluster identifier 114 that maps a subject's path to an existing behavioral cluster over the path space 112 using at least one path clustering criteria.

In an embodiment, mapping to a behavioral cluster is performed before the subject submitted the last version of the test.

In an embodiment, mapping to a behavioral cluster is performed when a certain criterion is met, for example, a certain number of versions of the solution have been submitted, a certain amount of time has passed, or the subject's solution matched a certain criteria, for example, compiled and passed a test.

The subject's path across solution abstraction clusters reflects the subject's behavior and progress in understanding the subject. In an embodiment, the method further comprises the step of preserving and maintaining metadata about subject's solution versions, solution version abstractions, for example, graphs, their mapping to solution abstraction clusters, the path of subject's solution version abstractions, for example, graphs, through solution abstraction clusters, and mapping of that path to a behavioral cluster.

At block 312, the step 300 includes mapping of the path of the subject into at least one behavioral cluster based on at least one path clustering criteria by the behavioral cluster identifier 114.

In an embodiment, the behavioral clusters are assigned at least one textual, color-coded, shape-coded or otherwise a descriptive label as identity markers. Some examples of descriptive labels include ‘a subject copied the solutions and does not understand the subject’, ‘a subject does not understand the subject and writes wrong code’, ‘a subject applied a different approach or approaches to solve the problem’, ‘a subject used one approach to solve the problem’, ‘a subject stayed in cluster for too long’, ‘a subject moves from one cluster to another often’, ‘most solutions of the subject lay in an incorrect cluster, but the correct solution is another less numerous cluster’, ‘a subject copied the solutions and does not understand the subject’, or “unknown”.

In an embodiment of the system 100, the clustering of paths into behavioral clusters is performed using either an expert artificial intelligence system or a machine learning artificial intelligence system trained on previously analyzed mapping of paths to behavioral clusters by the behavioral clusters identifier 114.

In an embodiment, the step of mapping of a subject's path to a behavioral cluster comprises the usage of at least one of a Density-Based Spatial Clustering of Applications with Noise (DBSCAN) algorithm or a k-nearest neighbors (KNN) algorithm.

In an embodiment, if the behavioral clusters identifier 114 fails to map a subject's path to an existing behavioral cluster, that path is used to form a new behavioral cluster.

In an embodiment, if the behavioral clusters identifier 114 fails to map a subject's path to an existing behavioral cluster, that path is classified as unknown or needing manual review by the instructor.

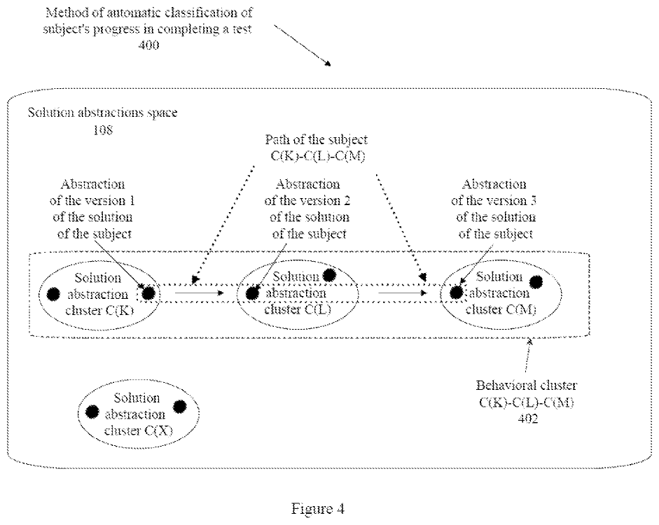

FIG. 4 illustrates a step 400 of path generation for each subject in a method for classification of study progress of multiple subjects in solving a complex problem, in accordance with an embodiment of the present disclosure. FIG. 4 illustrates a schematic view of the solution abstraction space 108, where a path of subject's solution version abstractions, for example, graphs, is depicted across three solution abstraction clusters. More specifically, the path C(K)-C(L)-C(M) of the subject is an ordered sequence of solution version abstractions, for example, graphs, 1, 2, and 3 of versions 1, 2, and 3 submitted by the subject, wherein the solution version abstractions, for example, graphs, 1, 2, and 3 are mapped to solution clusters C(K), C(L), and C(M) respectively. As seen in FIG. 4, the Path of subject C(K)-C(L)-C(M) is mapped to a Behavioral Cluster C(K)-C(L)-C(M) 402.

In an embodiment, a path of a subject does not have to be identical to other paths in the behavioral cluster to which it is mapped, but that relationship has to match a certain clustering criterion.

1. A method for automatically assigning, in a computer system, a behavioral category to a test subject's progress represented by an ordered collection of versions of a solution to a given test using solution graph clusters and behavioral clusters, the method comprising:
a. obtaining access to the test subject's collection of a version of a solution to the given test;
b. automatically generating a graph from the test subject's versions of the solution to the given test;
c. mapping each graph corresponding to a version of a solution, to a solutions graph cluster using a solution graph clustering criterion;
d. building a path for the test subject, wherein the path is an ordered sequence of at least one solution graph cluster to which at least one of the subject's solution version graphs belong based on at least one solution graph clustering criterion; and
e. mapping the subject's path to a previously identified behavioral cluster using at least one path clustering criteria.

2. The method of claim 1, wherein the solution graph clustering criteria is based on a metric defined over the space of solution graphs.

3. The method of claim 1, wherein the path clustering criteria is based on a metric defined over the space of paths.

4. The method of claim 1, where the step of mapping each solution version graph, to a solutions graph cluster using at least one solution graph clustering criteria further comprises using a machine learning artificial intelligence system trained on previously identified mapping of solution version graphs to solution graph clusters or an expert artificial intelligence system to map a solution version graphs to a solution graph cluster.

5. The method of claim 1, where the step of mapping the subject's path to a previously identified behavioral cluster using a e path clustering criteria further comprises using a machine learning artificial intelligence system trained on previously identified mapping of paths to behavioral clusters or an expert artificial intelligence system to map a path to a behavioral cluster.

6. The method of claim 1, where the step of mapping each solution version graph to a solutions graph cluster using a solution graph clustering criterion further comprises creating a new solution abstraction cluster for the solution version graph for which mapping could not be established or ignoring the solution version graph.

7. The method of claim 1, wherein the step of mapping the subject's path to a previously identified behavioral cluster using at least one path clustering criteria further comprises creating a new behavioral cluster for the path for which mapping could not be established or marking the path.

8. The method of claim 1, wherein the step of mapping of a solution version graph to a solution graph cluster includes a Density-Based Spatial Clustering of Applications with Noise (DBSCAN) algorithm or a k-nearest neighbors (KNN) algorithm.

9. The method of claim 1, wherein the step of mapping of a path to a behavioral cluster includes a Density-Based Spatial Clustering of Applications with Noise (DBSCAN) algorithm or a k-nearest neighbors (KNN) algorithm.

10. The method of claim 1, wherein the behavioral clusters are assigned at least one textual, color-coded, shape-coded or otherwise a descriptive label as identity markers.

11. A system for automatically assigning a behavioral category to a test subject's progress represented by an ordered collection of versions of a solution to a test using solution graph clusters and behavioral clusters, the system comprising:
a. a solution version collector configured to obtain access to the test subject's collection of versions of a solution to the test;
b. a solution version abstractor configured to automatically generate a graph from each of the test subject's versions of the solution to the test;
c. a solution abstraction cluster identifier configured to map each graph corresponding to a version of a solution, to a solutions graph cluster using a e solution graph clustering criteria;
d. a solution abstraction cluster tracker configured to build a path for the test subject, wherein the path is an ordered sequence of at least one solution graph cluster to which at least one of the subject's solution version graphs belong based on a solution graph clustering criteria; and
e. a behavioral cluster identifier configured to map the subject's path to a previously identified behavioral cluster using a path clustering criteria.

12. The system of claim 11, wherein the solution graph clustering criteria used by the solution abstraction cluster identifier is based on a metric defined over the space of solution graphs.

13. The system of claim 11, wherein the path clustering criteria used by the behavioral cluster identifier is based on a metric defined over the space of paths.

14. The system of claim 11, wherein the solution abstraction cluster identifier is further configured to use a machine learning artificial intelligence system trained on previously identified mapping of solution version graphs to solution graph clusters or an expert artificial intelligence system to map a solution version graph to a solution graph cluster.

15. The system of claim 11, wherein the behavioral cluster identifier is further configured to use a machine learning artificial intelligence system trained on previously identified mapping of paths to behavioral clusters or an expert artificial intelligence system to map a path to a behavioral cluster.

16. The system of claim 11, wherein the solution abstraction cluster identifier is further configured to create a new solution graph cluster for the solution version graph for which mapping could not be established or to ignore such solution version graph.

17. The system of claim 11, wherein the behavioral cluster identifier is further configured to create a new behavioral cluster for the path for which mapping could not be established or to mark the path.

18. The system of claim 11, wherein the solution abstraction cluster identifier is further configured to use a Density-Based Spatial Clustering of Applications with Noise (DBSCAN) algorithm or a k-nearest neighbors (KNN) algorithm.

19. The system of claim 11, wherein the behavioral cluster identifier is further configured to use a Density-Based Spatial Clustering of Applications with Noise (DBSCAN) algorithm or a k-nearest neighbors (KNN) algorithm.

20. The system of claim 11, wherein the behavioral cluster identifier is further configured to assign a descriptive label as identity markers to a subject's path based on the path mapping to a behavioral cluster.
